(19) United States
(12) Patent Application Publication
(10) Pub. No.: US 2024/0226405 A1
(43) Pub. Date: Jul. 11, 2024

(54) CANNULA FOR CONVEYING BODILY FLUIDS

(71) Applicant: Xenios AG, Heilbronn (DE)

(72) Inventor: Patrick Rauh, Freiburg (DE)

(21) Appl. No.: 18/042,746

(22) PCT Filed: Aug. 18, 2021
(86) PCT No.: PCT/EP2021/072948

§ 371 Date: Dec. 22, 2023

(30) Foreign Application Priority Data
Aug. 26, 2020 (DE) 10 2020 005 228.2

(51) Int. Cl. A61M 1/36

(52) U.S. Cl. CPC A61M 1/3659; A61M 1/3666; A61M 2202/0021; A61M 2202/0413; A61M 2206/20; A61M 2210/12

(57) ABSTRACT

A cannula for conveying body fluids includes a body extending in an axial direction from a proximal end to a distal end. The body defines a continuous inner cavity and includes at least two rows of holes in the distal region of the body, which are axially spaced apart from each other and each include at least two holes. The holes open in each row of holes from the inner cavity in the radial direction and are circumferentially spaced apart. A row of holes X is arranged distally to at least one further row of holes and a ratio of a total opening area of the holes of the row of holes X to the total opening area of the holes of the at least one further row of holes arranged in proximal direction to the row of holes X is 2:1 to 3:1.

### CROSS-REFERENCE TO RELATED APPLICATIONS

The present application is the national stage entry of International Application No. PCT/EP2021/072948, filed on Aug. 18, 2021, and claims priority to Application No. DE 102020005228.2, filed in the Federal Republic of Germany on Aug. 26, 2020, the disclosures of which are expressly incorporated herein in their entirety by reference thereto.

### TECHNICAL FIELD

The present disclosure relates to cannulas for conveying or pumping body fluids as well as circulatory support systems and corresponding methods, e.g., to prevent unfavorable pressure differences and/or volume flows.

### BACKGROUND

Cannulas can be inserted into the body of a patient for various medical applications to aspirate or remove body fluids and/or to deliver or administer therapeutic fluids to the patient. For example, cannulas may be intended for the gastrointestinal tract or the digestive tract, which allows excess and/or physiologically damaging fluids to be removed from the body, or for the local administration of drugs and/or nutrients. Furthermore, cannulas are used in particular for circulatory support, wherein low-oxygen and high carbon dioxide blood can be taken from the patient via a cannula and oxygen-rich and low carbon dioxide blood can be returned to the patient via the same or another cannula.

The oxygenation and especially the carbon dioxide reduction of the blood can thus be provided by extracorporeal life support systems, also known as ECLS (“extracorporeal life support”) or extracorporeal circulatory support systems, wherein extracorporeal membrane oxygenation takes place. In such a procedure, also known as ECMO (“extracorporeal membrane oxygenation”), the respiratory function of the patient is taken over by an external medical device. The lung function or respiration of the patient is thus at least partially replaced by such a medical device. Such a procedure can ensure oxygenation and in particular carbon dioxide reduction of the blood, for example if the patient's own respiratory function is or has to be suspended or suppressed in the event of cardiac surgery. In the case of lung diseases, such a procedure can also relieve the pressure on the lungs, allowing them to heal without exogenous ventilation for days or weeks, depending on the treatment.

As extracorporeal life-support systems, ECMO systems can hence be used, which include a membrane oxygenator and serve as gas exchanger for the patient's blood. The required cannulas are inserted into two blood vessels and the blood is continuously pumped through a membrane oxygenator, which replaces the gas exchange in the lungs. The blood can, for example, be taken from a venous access and returned via a venous or arterial access. Oxygenation then takes place, for example, by means of a veno-venous ECMO (VV-ECMO) or a veno-arterial ECMO (VA-ECMO).

For the removal of blood, prior art cannulas are described to exhibit a large number of holes at the cannula tip, wherein an extracorporeally arranged pump connected to the cannula via a blood hose or blood tube generates pressure differences between the holes and an outlet of the cannula by its pumping action to convey the blood. In order to allow a certain flexibility with regard to placement and therapeutic application, the holes are arranged in rows of holes, with the rows of holes being evenly spaced apart with only a small distance between them. In this way, the cannula can be inserted and placed according to the therapeutic approach, wherein a higher number of rows of holes allows blood to be drawn from the largest possible area of a blood vessel. Accordingly, in prior art solutions the number and dimensions of the holes are the same for each row of holes so that—regardless of the exact placement of the cannula—a comparable volume of blood can be collected along the cannula or the cannula tip.

One example of such a cannula with a substantially even arrangement of holes being evenly spaced apart and having the same size is known from WO 2016/022797 A1, wherein a large number of holes is present in the respective part of the cannula.

Further, cannulas are known from US 2007/0282243 A1, which are arranged to be inserted in the left ventricle and wherein a row of elongated holes at the distal end of the tip of the cannula provide for a maximum open surface. EP 3 656 414 A1 discloses cannulas with different evenly-spaced hole areas, wherein the hole size is continuously decreasing from the distal to the proximal end.

### SUMMARY

Based on the known state of the art, it is an aspect of the present invention to improve the pumping of body fluids and to prevent the occurrence of undesired pressure differences and volume flows as far as possible.

According to the present disclosure, it was recognized that due to the increased number and uniform dimensioning of the holes and the identical relative spacing of the rows of holes, pressure differences can occur which can be harmful to blood cells (for example due to shear forces) and thus cause undesired hemolysis. Such an undesirable effect can occur especially at holes that are located farthest away from the tip or closest to the pump. Furthermore, different volume flows can occur due to the increased number of rows of holes and their uniform spacing. At low volume flows, for example, the risk of local blood clotting events is increased in corresponding applications.

Furthermore, unfavorable turbulences can occur, which represent a further risk for the patient or impair the vascular structure and functionality of the vascular endothelium.

For example, even if a cannula according to the prior art is placed in the vena cava of a patient with the tip of the cannula at the level of the right atrium, the blood may not be sufficiently drained from the superior vena cava and adequate right ventricular relief cannot be ensured. It has been found that the volume flow at the tip of a cannula or in the region of the superior vena cava does not correspond to the typical flow physiological conditions of the volume flow from the superior or inferior vena cava or at the level of the right atrium. At the same time, too high pressure differences could be generated at the holes in the region of the inferior vena cava, which could lead to aspiration of the vessel wall. This results in an increased risk of local blood coagulation, possibly combined with an increased risk of increased hemolysis in the area of the inferior vena cava.

Accordingly, there is a need to improve the delivery or conveying of body fluids, taking into account the anatomy and physiology of the patient to be treated.

Accordingly, a cannula for the delivery or conveying of body fluids is suggested, which includes a tubular body extending in an axial direction from a proximal end to a distal end, the body defining a continuous inner cavity from the proximal end to the distal end and including at least two rows of holes in the distal region of the body. A “row of holes” is defined as a plurality of holes positioned circumferentially on the body, typically arranged in a cross-sectional area of the body. The at least two rows of holes are axially spaced apart from each other and each include at least two holes, wherein the holes in each row of holes open from the inner cavity in a radial direction and are spaced apart from each other in a circumferential direction. According to the disclosure, one row of holes X is arranged distally to at least one other row of holes. A ratio of a total opening area of the holes of row of holes X to the total opening area of the holes of at least one further row of holes arranged in the proximal direction to row of holes X is typically 2:1 to 3:1.

Several or all holes of the rows of holes proximal to the row of holes X can represent the total opening area, either individually or taken together, and which form the ratio of the total opening area of the row of holes X according to the disclosure. In this respect, at least one proximal row of holes can have a total opening area which is smaller, such that the ratio of the row of holes X to this one row of holes arranged proximally to the row of holes X is provided. When more than one row of holes arranged proximal to the row of holes X is considered, e.g. two such proximal rows of holes, the sum of the total opening areas of the two rows of holes to the total opening area of the row of holes X can thus form the ratio (while, for example, each of the two rows of holes has total opening areas which are so small that they lie outside of the range). Or, in any case, the holes of one of the two rows of holes, e.g., the row of holes immediately proximate and adjacent to the row of holes X, represent a total opening area whose ratio to the row of holes X is in the range. In such a case, the holes of the second row of holes may also have a total opening area in a ratio to the row of holes X or they may have a smaller total opening area which as such does not—in relation to the total opening area of the row of holes X—lie within the range.

An inner cavity of the body is to be understood as an inner lumen of the body which enables the conveyance of body fluids between the distal end or the distal region and the proximal end or the proximal region. The body and the inner cavity are typically elongated so that the axial direction is essentially a longitudinal direction of the body. The inner cavity and/or body may be circular, such that the body can be formed essentially as a tube wall. Alternatively, however, they can be asymmetrical, ellipsoidal and/or curved, at least in portions or sections. At the outer or most distal end of the cannula, a tip can also be provided, e.g., a rounded and/or atraumatic tip, which facilitates the insertion of the cannula into the body of a patient.

The terms “distal” and “proximal” are to be understood in such a way that “distal” indicates an orientation of the cannula with which the cannula is firstly inserted into the patient's body by the surgeon, i.e. with its tip, and “proximal” indicates an orientation in the opposite direction, which is facing the surgeon when using the cannula. In this way, an unambiguous orientation of the individual components of the cannula is made possible. From the point of view of the flow direction, wherein a flow direction from the proximal to the distal end is generally assumed, the terms “proximal” and “distal” correspond to each other when supplying body fluids, but when draining or removing body fluids from the patient's body they differ by reversing the flow direction. In the latter case, there is hence a flow from the distal end to the proximal end.

The “distal region” of the cannula body can also be defined as the area of the body that encompasses all rows of holes and can extend 50 to 60 cm from the distal end of the body in the proximal direction. However, depending on the application and the patient's anatomy, which may dictate the boundary conditions of the cannula to be used, the distal section may also extend 30 to 40 cm or 40 to 50 cm from the distal end of the body in the proximal direction. In other words, no further holes/rows of holes are provided proximal to the “distal region” for the delivery of body fluids. The “distal region” of the body thus defines the functionality of the cannula under application conditions by specifying the section or portion of the cannula where body fluid is to be delivered or received. Accordingly, this “distal region” can also be dimensioned smaller. In the case of applications for circulatory support, for example, it can also extend only about 10 cm to about 20 cm from the distal end of the body in the proximal direction.

The total opening area of the rows of holes is defined both by the number of holes and the shape or dimension of the holes per row of holes. The respective holes are designed to receive and/or release, e.g., body fluids when a pressure difference is applied between the proximal end and the distal end of the cannula, so that capillary action or even diffusion force is negligible or do not even occur.

In the circumferential direction, the at least two holes per row of holes are furthermore spaced apart from each other, so that they can be arranged asymmetrically, symmetrically or opposite to each other—for example in cross-section. If there are more than two holes per row of holes, however, it may be provided that at least two holes adjacent to each other have a relative distance in circumferential direction of, for example, less than the distance given by the circumference (divided by the number of holes per row of holes) in case of uniform spacing, e.g., a distance of less than half of the circumference divided by the number of holes per row of holes. For example, four holes may be provided, with the holes arranged as two pairs. If the spacing were uniform, the holes would be spaced about a quarter turn apart. However, the holes can also be arranged in such a way that two holes, each forming a pair, are less than an eighth of a turn apart. In this way, two pairs of holes are formed, whereby the pairs can be arranged symmetrically in the circumferential direction and on opposite sides and thus be spaced from each other by about half a revolution, but the holes of a respective pair are arranged closer to each other or adjacent. The arrangement of the holes can thus be uniform or asymmetrical and can be adapted to the application or the surrounding anatomical region in the inserted state of the cannula.

The ratio of the total opening areas also specifies that the total opening area of one or each row of holes arranged proximally to the row of holes X is smaller than the total opening area of the row of holes X. In some embodiments, the total opening area of each proximal row of holes or of all proximal rows of holes is smaller than the total opening area of the row of holes X, even if there are several rows of holes proximal to the row of holes X. As described above, the total opening area is determined by the number of holes as well as the dimensions of the holes. Due to the smaller total opening area of the at least one row of holes arranged proximal to the row of holes X, a pressure difference in the proximal region or portion of the distal region can be kept within a desired, flow-physiologically acceptable range. This also provides an improved pressure distribution between the rows of holes or for the at least one, proximal row of holes and row(s) of holes arranged distally thereto, such as row of holes X, at low peak pressure and with a more uniform volume flow. In the case of blood as a body fluid to be drained off, removed, or aspirated, the occurrence of hemolysis and also the risk of blood clotting can thus be considerably reduced or even prevented.

In some embodiments, the ratio of a total opening area of the holes in row of holes X to the total opening area of the row of holes immediately adjacent to row of holes X in the proximal direction is 2:1 to 3:1. In this way, pressure differences and volume flows between two immediately adjacent rows of holes can be kept within a predetermined range. Within such a range, the ratio can be selected for the individual application and/or for the physiological condition that occurs after catheterization of the cannula.

In some embodiments, the body includes exactly one row of holes in the proximal direction to row of holes X, which would then be the “proximal row of holes”. The only row of holes arranged proximally to the row of holes X in this case also has a total opening area which is smaller than the total opening area of the row of holes X and includes a ratio to the total opening area of the row of holes X. The total opening areas can also be selected in such a way that a limit of the above described ratio range is maintained. By providing only one row of holes arranged proximally to the row of holes X, an exact adjustment of the pressure differences and volume flows between the row of holes X and the proximal row of holes is achieved, i.e., for the entire region proximal to the row of holes X. By such a configuration only one single proximal row of holes hence has to be considered for the predefined or intended flow or flow conditions, when dimensioning the holes or the total opening area, respectively, which may reduce the number of (boundary) variables and may ensure a more stable delivery or conveyance.

For example, the total opening area of the row of holes X can be from about 35 mm² to about 45 mm², while the total opening area of the row of holes arranged in the proximal direction of row of holes X can be from about 10 mm² to about 20 mm². This improves the distribution of the proximal pressure difference so that the flow rate or delivery rate can be reduced accordingly and a more advantageous conveyance or delivery of body fluids can be provided. This also enables an improved volume flow of the row of holes X.

Such total opening areas, e.g., the ratio of these two total opening areas, are advantageous, if the cannula is adapted to drain body fluids such as blood and, if necessary, for use with a circulatory support system. For example, the distal area of the cannula can be placed approximately at the level of the right half of the heart and the cannula can accordingly drain blood from the vena cava of a patient. The total opening area of the row of holes X and the at least one row of holes proximal to the row of holes X can be formed or adapted primarily to collect and drain blood from the inferior vena cava. The blood to be drained or aspirated from the inferior vena cava includes approximately between 60 percent and 75 percent of the blood to be drained from the (inferior and superior) vena cava, while the remaining portion flows primarily from the superior vena cava. For example, depending on the patient, the cardiac output volume can be between about 4.0 L/min and about 5.5 L/min, so that the volume of blood to be drained from the inferior vena cava per unit time is typically between about 2.6 L/min and 3.6 L/min. The proportionally larger total opening area of the row of holes X results in a higher volume flow, or it is hence enabled that the flow rate may be reduced at the proximal row of holes closest to the pump. Accordingly, an improved volume flow distribution is provided, wherein the discharge or removal of the blood at least largely reduces the occurrence of hemolysis.

In addition to the at least one row of holes proximal to row of holes X, the body may further include at least one additional row of holes, for example one or two rows of holes, distal to row of holes X. In some embodiments, the body includes exactly one additional row of holes in the distal direction of row of holes X (which would then be the “distal row of holes”). This allows further flexibility in placing the cannula. The cannula can thus be used for applications wherein pumping or draining fluids distally of the row of holes X is advantageous from a flow physiological and/or anatomical point of view.

Accordingly, a plurality of rows of holes can be provided in the distal region of the body, wherein at least one row of holes is provided proximally to row of holes X. However, at least one additional row of holes can be provided both proximally and distally to row of holes X. Accordingly, the number of rows of holes in the body of the cannula is, e.g., two to five, three, or four. For example, the body can include three rows of holes.

For example, two rows of holes can be provided proximally to row of holes X and one row of holes can be provided distally to row of holes X, so that the total number of rows of holes is four. In an embodiment, only one row of holes is provided proximally and distally to row of holes X, so that the total number of rows of holes is three and row of holes X is arranged between the two rows of holes arranged proximally and distally.

Row of holes X can be predefined as a functionally central row of holes in the distal region of the body, regardless of the number of rows of holes. In other words, the holes of the row of holes X can be assigned a central function for conveying fluids or for dispensing and/or draining fluids in the radial region or on an outer surface of the body. Thus, even if the number of rows of holes should be greater than three and/or the number of rows of holes arranged proximally and distally to row of holes X should be different, row of holes X is of central importance from a functional point of view. This central importance may be determined not only by the total opening area of the row of holes X, but also by a dimensioning and/or an arrangement or an axial offset of the row of holes X with respect to the at least one other row of holes in the cannula body. In other words, the central function can be defined both by the total opening area and the relative arrangement in the distal region, which can, for example, be adapted to a specific application or may be anatomically predefined.

Accordingly, the row of holes X can be arranged and dimensioned for a specific application or a predefined anatomical region of a patient in order to best represent the desired cannula function in the inserted state. The at least one additional row of holes supports the delivery of body fluids. The ratio of the total opening area, which is predetermined according to the disclosure, further optimizes the compensation of pressure differences and the distribution of volume flows with regard to flow physiological tolerance ranges.

In order to further improve the adaptation of the total opening area to the physiological conditions of the anatomical conditions surrounding the distal region of the cannula in the inserted condition of the cannula, the number of holes per row of holes can be two to six holes, e.g., two to four holes. The row of holes X can be the “middle” row of holes, i.e. in the case of three or five rows of holes, the one row of holes being in the middle having a respective one (or two in the case of five rows of holes) row of holes distally and proximally to the row of holes X. In the case of four or six rows of holes, the row of holes X can be (when viewed from the distal end of the body) the row of holes in the penultimate or third-last position.

In this way, for example, pressure differences as well as any existing flow pattern of the fluids surrounding the distal region of the cannula can be taken into account. It can be advantageous if the number of holes in the respective rows of holes is different. The plurality of the holes further simplifies an alignment and adequate placement of the cannula by increasing the probability of adequate alignment of at least one hole in the targeted anatomical region or to the flow direction of a body fluid. By selecting an appropriate number of holes per row of holes under the conditions of use, an optimized and even pressure and flow distribution is provided with sufficient conveyance of body fluids.

In some embodiments, the number of holes per row of holes for at least two immediately adjacent rows of holes decreases from the distal end to the proximal end or is constant. This applies in particular to row of holes X compared to its immediately adjacent rows of holes in distal or proximal direction. If more than two rows of holes are provided, the number of holes can thus be different for each pair of immediately adjacent rows of holes, wherein the row of holes further distally arranged for each pair includes a larger or constant number of holes. The remaining, at least one row of holes may include a number of holes which corresponds to the number of holes of this distal row of holes or may also include a smaller or larger number of holes, regardless of the relative arrangement to the distal row of holes. However, the number of holes per row of holes for two immediately adjacent rows of holes in the cannula body can be decreasing and/or constant from the distal end to the proximal end.

Accordingly, the number of holes in each individual row of holes may be either constant with respect to or greater than its proximally adjacent row of holes. In other words, the number of holes per row of holes can successively increase in the proximal direction, but it can also be the same for two immediately adjacent rows of holes and be smaller, for example, for a row of holes further proximally and potentially subsequent rows of holes in the proximal direction.

The selection of the number of holes per row of holes, e.g., a decreasing number of holes per row of holes from the distal end to the proximal end of the body, can also be accompanied by an alternative dimensioning of the holes of each row of holes in order to define a predetermined total opening area of a more proximally arranged row of holes, which is either larger or smaller than the total area of a more distally arranged row of holes or constant with respect to the total area of the more distally arranged row of holes. For example, even with a smaller number of holes, a sufficient total opening area can be provided, for example, by correspondingly enlarging and/or changing the shape of the holes. Thus, the holes per row of holes and consequently the respective rows of holes can be adapted or configured for predefined pressure differences and volume flows.

The number of holes per row of holes is typically from two to six, e.g., from two to four. This applies in particular to the row of holes X. In an embodiment, row of holes X includes three holes. At least one of the at least one additional rows of holes arranged in the proximal direction of row of holes X, e.g., the row of holes arranged proximally and immediately adjacent to row of holes X, can include two holes. In an embodiment with one row of holes arranged proximally to the row of holes X, this row of holes can thus include two holes. This small number of holes in comparison to the row of holes X can considerably reduce unfavorable pressure differences and volume flows at the proximal end of the distal region or at the most proximal row of holes. This is generally advantageous from a flow physiological point of view and less harmful to the patient.

The number and, e.g., the dimensioning, of the holes of the cannula can be selected for a predefined delivery volume or delivery volume range in order to adapt to the anatomy and physiology of the patient in the distal area of the cannula. For example, the cannula can be used for circulatory support. When inserted, the cannula is located in the inferior vena cava and is adapted to drain blood from the inferior vena cava. The number and appropriate dimensioning of the holes per row of holes can prevent the occurrence of unfavorable volume flows that can cause hemolysis or reduce their occurrence as far as possible. In this respect, a number of two holes is particularly advantageous for the most proximal row of holes, which can also be the row of holes immediately adjacent to row of holes X, since the greatest pressure differences are typically observed in this cannula section or region. In other words, the total opening area of the most proximal row of holes can provide an improved volume flow distribution of the rows of holes distal to this row of holes and damage to the blood can be kept to a minimum due to the smaller number of holes in this row of holes.

The holes per row of holes can be spaced towards the distal end and in the axial direction, i.e., they each lie on a circle in the circumferential direction. In other words: the holes per row of holes can be evenly spaced from the distal end of the body in the longitudinal direction and arranged on a (circle) line in the circumferential direction, or the holes can essentially be located in a cross-section of the body. This also provides flexibility in placing and orienting the cannula. The manufacturing of the cannula can be simplified and the structural stability of the cannula can be improved. For example, in this embodiment the holes in the row of the circumferential direction may be located via rotating the cannula so that the holes are at a defined location and in a defined flow direction, wherein because of the even spacing towards the distal end, the body fluid is pumped via these holes. The location is very precise in view of the expected flow profile. This is on contrast to prior art cannulas, wherein the holes are generally continuously or evenly distributed along the circumference and/or wherein the holes are spiral-type or not located in rows but offset.

The shape of the holes can generally be adapted to the medical application and/or the flow rate of the body fluid or the desired total opening area. Accordingly, the holes can be essentially circular, ellipsoid, and/or oblong. Due to its shape, ellipsoids or oblong holes (advantageously with their long longitudinal axis axially oriented with the body) can be provided for a larger total opening area in view of the available circumference of the body, so that the axial extension of the body can be used as long as this is not detrimental to the medical application and the structural stability of the body.

The holes per row of holes may also be of different shapes. In some embodiments, the holes per row of holes have the same shape. This allows for easier handling of the cannula, especially since it does not require any orientation to be considered by the surgeon when placing the cannula. Due to the same or essentially identical shape, the total opening area for each hole per row of holes is the same, thus providing or at least enabling an even volume flow distribution. The manufacturing of the cannula is also simplified. Alternatively, however, it is also possible that, for example, if there is a large number and/or even number of holes per row of holes, different (e.g. two different) hole shapes are provided, which may be arranged alternately in the circumferential direction.

The holes in the body of the cannula can be of different shapes for at least two rows of holes. As described above, the holes can thus be adapted to the anatomical boundary conditions underlying the application purpose, and not least to interindividual anatomical characteristics of the patients, to which the distal region of the cannula is exposed in the inserted state. For example, different, physiologically determined volume flows based on the shape, dimensioning and/or number of holes per row of holes can be taken into account in this way.

In any case, the holes of the row of holes X can be differently shaped than the holes of at least one of the two rows of holes immediately adjacent to row of holes X, e.g., the proximally adjacent row of holes. The holes in the row of holes X can be formed as oblong holes. This allows a larger total opening area with respect to the circumference of the body, while at the same time maintaining the structural stability or integrity of the body and preventing or at least largely avoiding kinking during insertion or placement of the cannula.

Since their total opening area is smaller with respect to the row of holes X, the holes in at least one of the at least one further row of holes arranged in the proximal direction of the row of holes X can be correspondingly essentially circular, e.g., in the case of an unchanged number of holes. Thus, the at least one row of holes arranged proximally to the row of holes X may also have more holes than in the row of holes X, but each hole may have a smaller diameter, so that their total opening area is nevertheless smaller—despite an increased number of holes. Or a lower number of holes can be provided with a relatively larger diameter. The total opening area of such a row of holes arranged proximally to the row of holes X can nevertheless be smaller than that of the row of holes X in. In some embodiments, the row of holes adjacent to the row of holes X in the proximal direction includes circular holes, e.g., with the same or with a smaller number of holes compared to the number of holes of the row of holes X. Optionally, in this case only one row of holes proximal to the row of holes X is provided.

Furthermore, at least one row of holes can be provided distally to the row of holes X, e.g., a row of holes whose total opening area of the holes is in a ratio of 1:1 to 1:1.5 to the total opening area of the holes of the row of holes X.

In other words, the total opening area of the at least one row of holes arranged distal to the row of holes X is equal to or smaller than the total opening area of the row of holes X. The total opening area of the row of holes X is, thus, larger than the total opening area of the at least one distal row of holes and/or larger than the total opening area of the at least one proximal row of holes. Thereby, the total opening area of the at least one distal row of holes is in relation larger than the total opening area of the at least one, e.g., one, proximal row of holes. As described above for the at least one row of holes proximal to the row of holes X, this not only allows fine adjustment of the pressure differences and volume flows between the row of holes X and the at least one additional distal row of holes, but also in the entire end region of the cannula distal to the row of holes X or in the distal region of the cannula as a whole.

Due to the relations of the total opening areas and the orientation of the rows of holes, the central-functioning row of holes X can, compared to the at least one distal row of holes, effect a larger volume flow, which is beneficial from an anatomic and/or flow-physiological point of view. However, the at least one distal row of holes and the relation between the total opening areas ensure that body fluids are sufficiently pumped distal to the row of holes X, while the distal volume flow does not affect the volume flow via the row of holes X. In this way, different pathophysiological conditions and requirements of the body fluid to be pumped and the corresponding anatomic target region may be considered. Such an embodiment is, thus, of advantage in comparison to prior art cannulas, wherein the holes are generally continuously or evenly distributed along the circumference and are of equal dimensions or wherein the total opening area of the holes at the distal end of the cannula is always the maximum.

The total opening area of the row of holes X can, for example, be from about 35 mm² to about 45 mm², while the total opening area of the row of holes arranged in the distal direction of the row of holes X can be from about 30 mm² to about 40 mm². Compared to the at least one row of holes proximal to the row of holes X, a larger total opening area is thus provided in the at least one row of holes distal to the row of holes X, so that a sufficient volume flow can be provided even with a lower pressure difference and/or surrounding flow rate of body fluids.

The exemplary range of this total opening area is particularly advantageous if the cannula, as described above, is to be designed for use with a circulatory support system and the distal region of the cannula is to be placed, for example, at the level of the right half of the heart, so that the cannula can accordingly drain or remove blood from the lower/upper vena cava of a patient. The total opening area of the at least one row of holes distal to row of holes X can be primarily configured to collect and drain blood from the superior vena cava. The total blood to be drained from the superior vena cava accounts for between 20 percent and 40 percent of the blood to be drained from the vena cava. The total volume of blood to be drained from the superior vena cava at the corresponding cardiac output volume is, for example, from about 1.4 L/min to about 1.9 L/min per time unit.

In some embodiments, the at least one row of holes arranged distally to the row of holes X includes three, four or five, e.g., four holes. They can be of any shape, e.g., essentially circular holes. For example, a distal row of holes can be provided with four holes, which are circular in shape. Both the shape, including the radius, and the number of holes are adapted to the intended total opening area. The holes arranged distally to the row of holes X can thus be smaller than those in the row of holes X. They can be distributed evenly, i.e., evenly spaced, over the circumference of the body in the circumferential direction. This further simplifies orientation and placement of the cannula and provides improved delivery or conveyance even at the distal end of the body.

The holes are also adapted for the delivery and/or absorption of body fluids, wherein potential vortices and turbulence can be prevented by the shape of the holes. Accordingly, the holes may be rounded towards the outer surface of the body and/or towards an inner wall of the body defining the cavity to avoid a sharp-edged profile. For example, the holes may be laser-cut and optionally include an inner, e.g., seamless, edge for structural reinforcement. This further improves the structural stability or integrity. This eliminates the need for conventional wire reinforcement in the area of each hole, resulting in improved flexibility and reduced risk to the patient, for example, from wire breakage and/or protruding wires. Furthermore, the rounded shape significantly reduces shear forces or shear stress, which could mechanically affect the body fluid, especially blood cells, when in contact with the surface of the cannula.

Depending on the area of application of the cannula, the holes in a row of holes can be arranged in different ways. Thus, the holes of at least one row of holes can be arranged symmetrically or asymmetrically in relation to the longitudinal axis of the body. In other words, the holes can be arranged mirror-symmetrically or with an offset. While a symmetrical arrangement facilitates the placement and orientation of the cannula, an asymmetrical arrangement can take into account a predetermined or specific volume flow of body fluids that surround the distal part of the body in the inserted state of the cannula (which may flow from a predetermined direction). All rows of holes can be adapted to each other. The holes of each row of holes can optionally be arranged symmetrically. In some embodiments, the holes of different rows of holes are adapted to each other in the longitudinal direction so that if the number of holes in one row of holes is small (compared to another row of holes, e.g., row of holes X), e.g. in the case of a row of holes with two holes and another row of holes with four holes, there is no offset of the holes (in the circumferential direction) of the two rows of holes, which could potentially complicate placement and/or orientation of the cannula. Accordingly, the holes of rows of holes with an equal number of holes can be arranged in such a way that they are circumferentially located at a similar position on the body, wherein the position can also be determined by a center or center of gravity of the holes, if the holes between the respective rows of holes have a different opening area, for example. Conversely, the holes of a row of holes with an odd number of holes, for example three holes, may be arranged circumferentially with an offset to holes of other rows of holes with an even number of holes, for example two or four holes.

To further facilitate cannula orientation and placement and to allow for uniform delivery or absorption of body fluids in different directions, the holes of at least one row of holes are evenly spaced apart along the circumference. In some embodiments, the holes of all rows of holes are equally spaced from each other in the circumferential direction and also the holes of different rows of holes (in the longitudinal direction) are adapted to or aligned with each other with regard to their respective positioning. A symmetrical arrangement in the circumferential direction with respect to the longitudinal axis of the body can also include holes which are arranged at irregular or different distances from each other, for example two pairs of adjacent holes of a row of holes, wherein the pairs of holes are arranged in the radial direction on opposite sides of the body, as described in the above. However, some embodiments include complete symmetry with even spacing of the holes of a row of holes with a symmetrical opening to the inner cavity.

In order to further adapt the rows of holes to the anatomy and therapeutic purpose, the spacing of the rows of holes per cannula may vary. In some embodiments, the body has at least three rows of holes, with each pair of adjacent rows of holes being spaced differently in the axial direction than any other pair of adjacent rows of holes. Thus, in addition to the row of holes X and a row of holes arranged proximally thereto, a row of holes arranged distally to the row of holes X may be provided, wherein the distance between the distal row of holes and the row of holes X differs from the distance between the row of holes X and the row of holes proximal to the row of holes X, i.e., the distances are larger or smaller in each case.

For example, the distance between the row of holes X and the at least one row of holes arranged in proximal direction to the row of holes X, e.g., the immediately adjacent row of holes, may be about 45 mm to about 60 mm. A ratio of this distance to the distance of the row of holes X and the distal end of the body can cover a range from 1:1.2 to 1:1.4.

The distance between the row of holes X and the distal end of the body can be about 60 mm to about 80 mm. A ratio of this distance to the distance of the most proximal row of holes and the distal end of the body can range from 1:1.65 to 1:1.85.

Accordingly, the distance between the row of holes X and a proximal row of holes, e.g., a most proximal row of holes, may be less than the distance between the row of holes X and the distal end of the body. However, the ratio is only given if the body extends far enough distal to the row of holes X. Otherwise, the distance of the proximal row of holes to the row of holes X would have to be selected as absolute distance.

In some embodiments, the body also includes at least one further row of holes, e.g., a single row of holes, that is arranged distally to row of holes X, wherein the distance between the row of holes X and the further row of holes arranged adjacent to the row of holes X in the distal direction is about 28 mm to about 43 mm. A ratio of this distance to the distance of the row of holes X and the distal end of the body can be in the range of 1:1.8 to 1:2.2.

As described above, the distance between the distal row of holes immediately adjacent to the row of holes X and the row of holes X can be smaller than the distance between the row of holes X and the row of holes immediately adjacent to row of holes X in the proximal direction. Although the above ratios and absolute distances are not limited to a specific application, they are particularly advantageous when used to support a patient's circulation. Under such application conditions, the cannula is typically placed in the inferior vena cava and at the level of the right side of the heart. In this way, by arranging the rows of holes and applying suction pressure from a pump connected to the cannula, volume flows are achieved that correspond to the physiological conditions in the vena cava so that the row of holes X and the row(s) of holes proximal to the row of holes X drain the blood to be drained from the inferior vena cava, which includes between 60 and 75 percent of the blood to be drained from the vena cava. An optional row of holes distal to the row of holes X and/or an optional opening at the distal end of the body can drain additional blood flow from the superior vena cava.

In addition to the rows of holes, the body can typically have at least one opening at the distal end that opens in the axial direction. In some embodiments, the distal end has an opening that essentially corresponds to the cross-sectional area of the cavity. The opening, which can be located at the tip of the cannula, thus provides an additional means for receiving and/or delivering body fluids. This can be advantageous if the cannula, when inserted, is oriented in such a way that body fluids such as blood flow essentially in the axial direction of the body or are to be delivered or conveyed in this direction, for example, if the cannula is configured and used for circulatory support and has been placed at the level of the right half of the heart. An opening that is essentially the same size as the cross-sectional area of the cavity also has the advantage of providing the least possible flow resistance and significantly reduces the risk of vortices and/or turbulence. Furthermore, the opening that opens in the axial direction can serve as a back-up in case, for example, one or more holes of the rows of holes are sucked against a patient's vessel wall.

The body of the cannula also has an outlet. The outlet can be defined by a circular opening at the proximal end of the body, for example. Furthermore, such a total opening area is also advantageous for delivery of body fluids, such that physiological limits can be maintained within the cannula and also when delivering body fluids via the holes. Accordingly, the outlet can also be an inlet of the cannula under changed conditions of use, e.g. to supply oxygen-enriched blood.

In order to further improve the pressure differences and the flow rate at the at least one row of holes located distal to the row of holes X, the total opening area of the holes can be dimensioned in such a way that it is larger than the outlet area, which can be coupled with a pump, for example. Accordingly, the ratio of the total opening area of the holes to the cross-sectional area of the cavity at the outlet can be 1.5:1 to 2:1.

In an embodiment for collection or removal of body fluids, the opening and the holes may together define an inlet (otherwise, namely when body fluids are supplied, the opening at the distal end and the holes represent the outlet) and the proximal end of the body an outlet (or, otherwise, an inlet), e.g., with a ratio of the total opening area of the inlet (outlet) to the cross-sectional area of the cavity at the outlet (inlet) ranging from 1.5:1 to 2.5:1.

For example, the total opening area of the inlet or inlet region can be about 120 mm² to about 150 mm² while the total opening area of the outlet is e.g. about 60 mm² to 80 mm².

For improved flow conditions, especially if the cannula is to be used to drain blood from the vena cava to support circulation, the total opening area of the respective rows of holes can furthermore be in a predetermined ratio to the cross-sectional area of the cavity at the distal end of the body, such that anatomical and physiological flow conditions at the location of the positioning of the rows of holes in the body vessel can hence be taken into account.

Accordingly, the total opening area of row of holes X can be 85% to 95% of the cross-sectional area of the cavity at the distal end of the body. The distal end can be regarded as a tip area and can optionally have an opening that allows axial withdrawal or discharge of body fluids, e.g., blood. The opening at the distal end will typically have a cross-sectional area equal to the cross-sectional area of the cavity. In specific applications, the opening at the distal end could also be smaller than the cross-sectional area of the cavity. The cross section of the body and the cavity may be essentially constant along the longitudinal axis or in the axial direction of the body, but may also increase in sections in the proximal direction or may continuously increase over the entire length of the body or as a specific position on the body. For example, the cross-section may be 40 mm² to 50 mm², such that the total opening area of the row of holes X may be about 34 mm² to about 48 mm² under such circumstances.

Also, the total opening area of the at least one row of holes proximal to row of holes X, e.g., its proximally adjacent row of holes, can be 30% to 40% of the cross-sectional area of the cavity at the distal end of the body. With an exemplary cross-sectional area of about 40 mm² to about 50 mm² described above, the total opening area of the row of holes X arranged proximally to the row of holes X can be about 10 mm² to about 20 mm², e.g., about 12 mm² to about 18 mm².

Furthermore, the body can include at least one additional row of holes, as described above, which is arranged distally to the row of holes X. The total opening area of the at least one row of holes arranged distally to the row of holes X, e.g., one distal row of holes, can be 75% to 85% of the cross-sectional area of the cavity at the distal end of the body. With an exemplary cross-sectional area of about 40 mm² to about 50 mm² of the cavity described above, the total opening area of the at least one distal row of holes arranged distally to the row of holes X can be about 30 mm² to about 42 mm², e.g., about 32 mm² to about 38 mm².

The selected total opening areas of the respective rows of holes can provide predetermined volume flow ratios, which are particularly advantageous for circulatory support. Suction pressures can thus be provided in the row of holes X and, if necessary, in the at least one row of holes arranged distally to the row of holes X, which enable an improved volume flow in the respective row of holes and in the corresponding section or portion of the body. At the same time, sufficient pressure remains at the one or more proximal rows of holes to ensure adequate delivery of body fluids in this section of the body as well, wherein unfavorable peak pressures and volume flows at the one or more proximal rows of holes are reduced so that the level of the flow rate at the corresponding holes can be kept within a physiological limit. When the cannula is placed in the vena cava, e.g., with the distal region at the level of the right half of the heart, the efficiency of blood collection or withdrawal can be significantly improved, such that the collection of blood essentially corresponds to physiologically provided volume flows.

For example, the row of holes X and at least one proximally arranged row of holes can be dimensioned in such a way that it can receive between 60% and 70% of the total blood flow (“total volume flow”) in the vena cava and discharge or conduct it via the body of the cannula. In this way, for example, about 40% of the volume flow of the cannula could be discharged via the row of holes X and about 30% of the volume flow of the cannula via at least one row of holes proximal to it. The total volume flow discharged through the row of holes X and the at least one proximal row of holes would then correspond to the physiologically typical volume flow in the inferior vena cava of about 65% of the total volume flow in the vena cava. If the cannula is appropriately placed in the anatomical target region of an arrangement of the row of holes X at the level of the right atrium and at least one further row of holes proximal to it in the inferior vena cava, the cannula would adequately represent the flow physiological boundary condition. In an advantageous configuration of the body with at least one, e.g., one, distal row of holes and an opening at the distal end of the body, the opening and the corresponding holes can absorb or receive the remaining approximately 30% to 40% of the total volume flow present in the vena cava and discharge it via the body of the cannula. This would correspond to the typical volume flow in the superior vena cava of about 35% of the total volume flow. Again, the at least one distal row of holes, which, if adequately positioned in the superior vena cava, would be able to proportionally receive and discharge the corresponding physiological blood flow. For example, about 15% of the cannula's delivery volume can be discharged via the distal row of holes and the opening in equal parts. The respective portion of the total volume flow of the at least one distal row of holes may, e.g., be provided by a ratio of the total area between the at least one distal row of holes and the row of holes X, wherein the total opening area of the at least one distal row of holes is smaller than of the row of holes X.

The number of rows of holes, the number of holes, the spacing of the rows of holes, the arrangement of the holes, the shape of the holes and/or the dimensioning of the holes can thus be adapted to an application and for given fluidic and anatomical conditions. Thus, cannulas can be provided for any application, wherein each cannula is formed and/or configured for an application with a given tolerance range.

In contrast to prior art cannulas with a high number of rows of holes, which are evenly spaced from each other and each of which has a correspondingly high number of holes, which are furthermore also evenly dimensioned, the cannula to be used for a given application can even be configured individually for each patient, especially in the light of the patient-specific, anatomical properties and the dimensions of the vena cava before the right atrium, such that an optimal transportation of fluids can be provided with an optimized pressure and flow distribution.

The cannula may be configured or formed, e.g., to drain body fluids, e.g., blood, via an outlet at the proximal end of the body. Under such circumstances, the proximal end of the body can define an outlet which can be fluidically coupled with a circulatory support system, e.g., an oxygenator and/or a pump.

In order to support the circulation, especially blood that is low in oxygen and rich in carbon dioxide can be withdrawn from the patient by means of the cannula. Accordingly, when the cannula is inserted, the distal area can be designed to drain blood from the blood circulation, e.g., from the atrium of the right half of the patient's heart. In some embodiments, the cannula is designed in such a way that, when inserted, it lies essentially in the inferior vena cava, wherein the row of holes X is arranged in such a way that the holes of the row of holes X open at the level of the right atrium. Further distally arranged rows of holes may protrude into the superior vena cava. Although blood collection or withdrawal through the row of holes X and at least one row of holes proximal to the row of holes X may be sufficient for circulatory support, the distal row(s) may also provide improved blood collection from the superior vena cava without impairing the collection from the inferior vena cava and/or right atrium. Further support for upper vena cava blood collection may be provided through an opening at the distal end of the body as described above.

Accordingly, the cannula can be placed so that blood present in or flowing into the right atrium can be effectively collected through the holes in row of holes X. Furthermore, with such placement and orientation of the row of holes X, blood can also be drawn from the inferior vena cava through both the row of holes X and the at least one row of holes proximal to the row of holes X. In some embodiments, the at least one further row of holes arranged proximally to row of holes X is arranged in such a way that the holes of the at least one further row of holes arranged proximally to row X open at the level of the inferior vena cava when the cannula is inserted.

The relative arrangement of these rows of holes, which can be provided, for example, by the exemplary spacing described above, allows for an advantageous blood collection from the vena cava, especially the inferior vena cava or the ratio between the superior and inferior vena cava, wherein the ratio of the total opening areas provides an improved flow distribution and undesirable pressure differences can be avoided at the at least one row of holes arranged proximally to the row of holes X and advantageous volume flows can be provided for the row of holes X and potentially the at least one row of holes arranged distally to the row of holes X.

The above aspect is further achieved by a circulatory support system, which includes a cannula as described herein. The circulatory support system can include at least one blood pump, which can be coupled, for example, by means of a blood tube to an outlet at the proximal end of the body and by means of which blood can be taken from the distal region and led or conducted to a gas exchanger, such as a membrane oxygenator, and then returned to the patient. Accordingly, the circulatory support system may also include a gas exchanger. To supply the oxygen-enriched and low-carbon dioxide blood, an additional cannula may be provided in the circulatory support system, which is fluidically connected to the extracorporeal circulation. This further cannula for recirculation may also be a second cannula as described herein. Due to the different functionalities of the cannulas in blood collection and blood supply, the two cannulas of the circulatory support system may in turn have different configurations. Alternatively, however, the cannula can also be designed as part of a double cannula or a cannula with double lumen and be coupled at the proximal end, for example. In this way, veno-venous ECMO (VV-ECMO) can be performed, for example.

Accordingly, the cannula can be used for circulatory support of a patient.

The above aspect is further achieved by a method using a cannula as described herein. The method includes at least:
inserting the cannula through an inguinal vein of the patient;
advancing the cannula such that the distal portion of the cannula is advanced into the inferior vena cava; and
placing the cannula so that the row of holes X is positioned at the level of the right half of the heart.

Accordingly, the cannula can typically be inserted into the patient via an inguinal or femoral vein and advanced so that it is positioned in the patient's vena cava, thus allowing fluid communication between the right half of the heart and a circulatory support system. In some embodiments, the cannula is placed in such a way that the holes of row of holes X open at the level of the right atrium when the cannula is inserted. For example, the cannula can be placed in such a way that the holes of the at least one row of holes arranged proximally to the row of holes X open at the level of the inferior vena cava, when the cannula is inserted. In this way, for example, an improved collection of blood from both the right atrium and the inferior vena cava can be provided, both due to the predefined total opening areas and the relative arrangement of the row of holes X and the at least one row of holes arranged proximally to the row of holes X. After positioning the cannula approximately at the level of the right atrium, the method allows the collection of venous patient blood. In a further method step, the blood can be fed extracorporeally to a gas exchanger in order to increase its O₂ partial pressure and/or to reduce CO₂ in the collected blood. The blood processed in this way in a gas exchanger, e.g., using a heating device to maintain the body temperature of the blood, can be returned to the patient, e.g., by means of a further cannula. This additional cannula may or may not be of the type described herein. The cannula may also be of the double cannula type, in which case at least one lumen is provided by a cannula as described herein. Therefore, the method using the cannula described herein can be used, for example, for blood collection, blood processing, especially for pulmonary support of a lung patient, for example, as a result of pneumonia or COPD, and subsequent blood return to the patient.

The body can include at least one row of holes distal to the row of holes X, wherein the cannula is placed in such a way that the holes of the at least one row of holes distal to the row of holes X open at the level of the superior vena cava, when the cannula is inserted. In this way, for example, in addition to the blood collection from the right atrium and the inferior vena cava, an improved blood collection from the superior vena cava is also provided, so that the total volume of blood that is collected from the patient, for example, can be increased and can correspond to the physiological conditions between the superior vena cava and the inferior vena cava.

Although the cannula has been described above specifically for blood collection and circulatory support applications, it may also be configured to deliver blood or to collect or dispense alternative body fluids, for example, in the gastrointestinal tract or to aspirate fluids from the pulmonary system.

### BRIEF DESCRIPTION OF THE DRAWINGS

Further embodiments are explained in more detail in the following description of the Figures, in which:

FIG. 1A is a schematic side view of a cannula with row of holes and a distal row of holes.

FIG. 1B is a schematic cross-section of the cannula according to FIG. 1A along the line of intersection A-A.

FIG. 1C is a schematic cross-section of the cannula as shown in FIG. 1A along the line of intersection B-B.

FIG. 2 is a schematic representation of a further embodiment of the cannula shown in FIG. 1A.

FIG. 3 is a schematic representation of a further embodiment of the cannula shown in FIG. 1A.

FIG. 4 is a schematic representation of a cannula with inlet and outlet areas.

### DETAILED DESCRIPTION

In the following, embodiments will be explained in more detail with reference to the accompanying Figures. In the Figures, corresponding, similar, or like elements are denoted by identical reference numerals and repeated description thereof may be omitted in order to avoid redundancies.

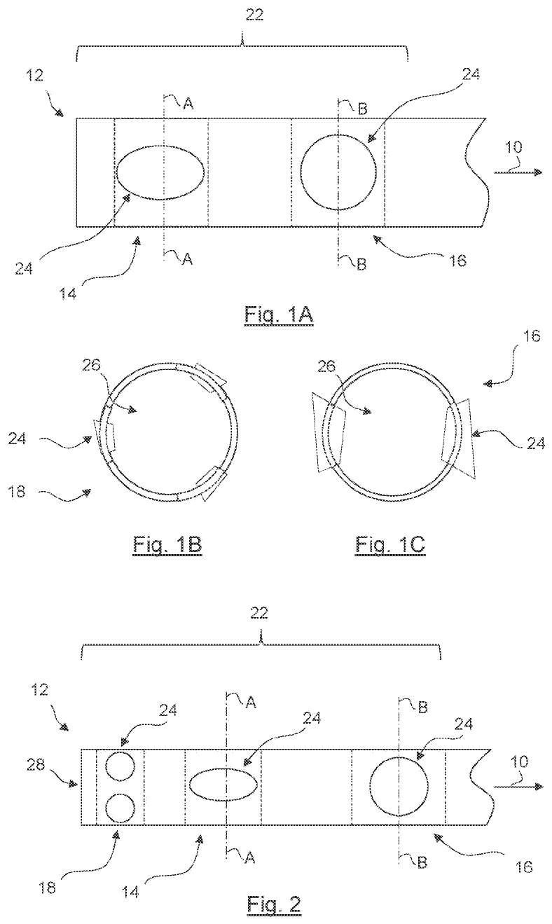

FIG. 1A schematically shows a side view of a cannula, with a body being elongated and extending axially. The body is substantially tubular or circular in cross-section and extends from a proximal end (not shown) to a distal end 12 of the body, wherein the terms “distal” and “proximal”, as described above, are to be understood such that “distal” means an orientation away from the surgeon and “proximal” means an orientation towards the surgeon with respect to the insertion and placement of the cannula into a patient. In a distal region 22 of the body, which extends from the distal end 12 in the proximal direction 10 and includes all rows of holes as shown by the arrow, there are two rows of holes 14, 16, as indicated by the dotted areas, which each include a plurality of holes 24 (only one hole per row of holes shown in FIG. 1A).

One row of holes 14 (e.g., a row of holes X described above) is provided in the distal area 22 and forms a central row of holes and is formed or configured in such a way that, when the cannula is inserted, the greatest possible volume flow can be conveyed, i.e., supplied or removed, via this row of holes 14. For example, the cannula can be designed for circulatory support and for draining blood from the blood circulation of a patient, wherein the row of holes 14 is dimensioned and arranged in such a way that it can drain blood from the upper and lower extremities or the upper and lower vena cava and/or from the right atrium of the patient. However, alternative applications may also be provided, and the cannula may be designed accordingly. In such cases, the row of holes 14 is also used to provide central volume transport. It can thus serve as an anatomical orientation when placing the cannula, depending on the anatomical and flow physiological boundary conditions.

In this example (FIG. 1B), the row of holes 14 includes three holes 24, which are formed as oblong holes in order to provide the largest possible opening area with regard to the available circumference and the structural stability of the body. In other words, a wall or surface of the body is not only used in the circumferential direction, but also in the longitudinal direction. Although this is not shown in the side view depicted in FIG. 1A, the holes 24 in the row of holes 14 in this example are evenly spaced in a circumferential direction and symmetrically arranged relative to the longitudinal axis of the body, thereby facilitating cannula placement and orientation and allowing for the even distribution of body fluids such as blood to be received from the surrounding environment.

The arrangement of row of holes 14 is particularly shown in FIG. 1B. Accordingly, it can be seen that the body defines an inner cavity 26, which extends from the distal end 12 in the proximal direction 10, with holes 24 providing a fluidic connection between the cavity 26 and the surrounding or external surface of the body.

Another row of holes 16 is located proximal to row of holes 14. The proximal row of holes 16 includes two holes 24, which are also evenly spaced apart from each other in the circumferential direction and are symmetrically arranged in view of the longitudinal axis of the body, i.e., facing or opposite to each other. The holes 24 of the proximal row of holes 16 are circular. However, since the holes 24 of the proximal row of holes 16 do not extend in the axial direction or are not formed as ellipsoids, the total opening area of these holes 24 is smaller than the total opening area of the holes 24 of row of holes 14, even if the diameter is (optionally) larger. In the present exemplary embodiment, a ratio of the total opening area of holes 24 of row of holes 14 to the total opening area of holes 24 of proximal row of holes 16 is 2:1 to 3:1. This is illustrated by FIGS. 1B and 1C, wherein FIG. 1C shows a cross-section along the intersection line B-B or through row of holes 16. For example, this ratio of the total opening area of holes 24 of the row of holes 14 to the total opening area of holes 24 of the proximal row of holes 16 is 2.3:1 to 2.9:1 or, e.g., about 2.6:1.

This ratio enables an improved pressure and flow rate distribution so that no undesirable pressure differences are provided at the proximal row of holes 16 and physiological limits are not exceeded. At the same time, an improved volume flow is enabled at the row of holes 14. In order to enable an even more uniform flow and prevent the occurrence of turbulence, the holes 24 of the respective rows of holes 14, 16 are essentially equally spaced towards the distal end 12 and in the axial direction. Also, the holes 24 are rounded towards the outer surface of the body and the inner wall of the body defining cavity 26, such that any flow at the opening surface of the holes 24 can be reduced or even avoided. Sharp-edged end regions can be avoided, such that the occurrence of hemolysis due to mechanical destruction of blood cells is considerably reduced when draining blood.

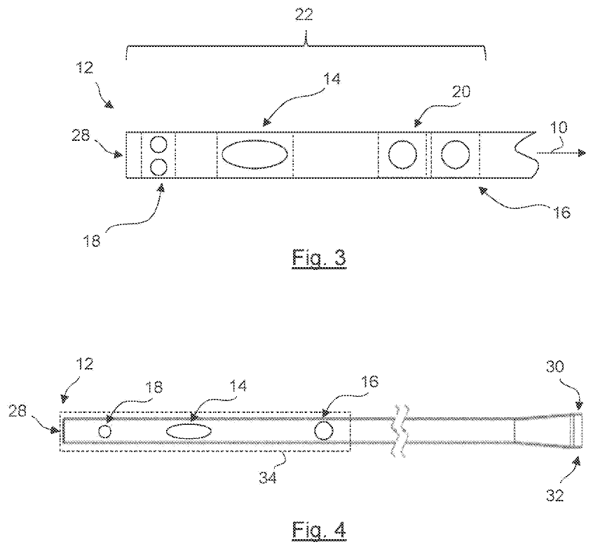

FIG. 2 schematically depicts another embodiment of the cannula as shown in FIG. 1A, with the rows of holes 14 and 16 corresponding to the design as shown in FIGS. 1A to 1C. In this embodiment, an additional row of holes 18 is provided, which is arranged distally to the row of holes 14 and the row of holes 16. In this distal row of holes 18, four holes 24 are provided (only two holes are shown in FIG. 2), which are circular in shape and also equally spaced from each other in the circumferential direction, but are smaller in size in comparison to the holes in the rows of holes 14 and 16. In this way, a total opening area is provided which is suitable for conveying body fluids at the distal end 12 of the body. The ratio of the total opening area of holes 24 in the distal row of holes 18 to the total opening area of holes 24 in the row of holes 14 is advantageously 1:1 to 1:1.5. Thereby, blood can be effectively withdrawn from the superior vena cava, for example, without the distal row of holes 18 and row of holes 14 negatively influencing each other.

In this configuration, the row of holes 14 forms a row of holes which is positioned in the middle, enclosed by two further rows of holes, such that when draining body fluids and from the distal end 12 in the proximal direction 10, the distal row of holes 18 defines a first row of holes, row of holes 14 a second, central row of holes, and the proximal row of holes 16 a third row of holes. The number of holes 24 per row of holes 18, 14, 16 for each adjacent row of holes decreases from the distal end 12 to the proximal end or the number of holes 24 in the distal row of holes and the row of holes 14 is greater than the number of holes 24 in the respective proximal row of holes 14, 16.

In addition to the optimized ratio of the total opening areas of the rows of holes 14, 16, 18 to each other, each pair of adjacent rows of holes 14, 16, 18 is spaced differently in the axial direction than any other pair of adjacent rows of holes 14, 16, 18. In some embodiments, the spacing of the distal row of holes 18 and the row of holes 14 is smaller than the spacing of the row of holes 14 and the proximal row of holes 16. In this way, not only can predetermined pressure and flow conditions be maintained or provided. Such a different arrangement also allows the distal area 22 to be tailored or adapted to the respective application. For example, holes 24 of the row of holes 14 can open at the level of the right atrium when the cannula is inserted, wherein holes 24 of the distal row of holes 18 open at the level of the superior vena cava and holes 24 of the proximal row of holes 16 open at the level of the inferior vena cava.

For such an application, the distance between the row of holes 14 and the proximal row of holes 16 can be about 45 mm to about 60 mm, wherein the exact distance can be adapted to the anatomical characteristics of the patient and, for example, different cannulas with different distances and/or dimensions of the rows of holes can be used for different patient groups. Accordingly, the distance between the row of holes 14 and the distal end 12 of the body can also be about 60 mm to about 80 mm. The distance between the row of holes 14 and the distal row of holes 18 can be about 28 mm to about 43 mm.

Furthermore, an opening 28 is provided at the distal end 12 of the body, which opens in the axial direction and essentially corresponds to the cross-sectional area of cavity 26, as it is dimensioned, for example, in FIG. 1B at the level of the row of holes 14. This allows blood to flow into the cannula from the axial direction or in the flow direction of a blood vessel, for example, such that the occurrence of vortices is further reduced. Although such an opening 28 is not shown in the design shown in FIG. 1A, it can optionally be provided in this configuration and opening 28 is also optional for the configuration shown in FIG. 2.

FIG. 3 schematically illustrates a further embodiment of the cannula as shown in FIGS. 1A and 2, wherein the cannula differs from the embodiment shown in FIG. 2 in that a further proximal row of holes 20 is provided, which is arranged between the proximal row of holes 16 and the row of holes 14. The additional proximal row of holes 20 enables an alternative pressure and flow rate distribution, which can be advantageous for given applications. The ratio of the total opening area of the holes 24 in the row of holes 14 to the total opening area of the holes in the distal rows of holes 16, 20 is still 2:1 to 3:1, wherein the respective diameters and opening areas of the holes in these distal rows of holes 16, 20 are smaller than those of the holes 24 in the proximal row of holes 16 as shown in FIG. 2. However, the distal rows of holes 16, 20 still contain two holes 24 each, which are arranged on opposite sides of the body.

Furthermore, the rows of holes 14, 16, 18, 20 are also arranged according to this embodiment in such a way that each pair of adjacent rows of holes 14, 16, 18, 20 is spaced differently in the axial direction than any other pair of adjacent rows of holes 14, 16, 18, 20. In other words, the advantageous ratios of the total opening areas and the arrangement of the rows of holes 14, 16, 18, 20 to each other are also realized in this design.

FIG. 4 shows a schematic representation of a cannula, which essentially corresponds to the embodiment shown in FIG. 2. In this embodiment, an inlet 34 or an inlet area and an outlet 32 are also provided. The inlet 34 is provided by the holes of the respective rows of holes 14, 16, 18 in the distal area and by the opening 28 at the distal end of the body. Accordingly, an outlet is provided at the proximal end 30, which can be fluidically coupled to a pump of a circulatory support system, for example.

The total opening areas of the rows of holes 14, 16, 18, which can be dimensioned and sized, for example, as a ratio with respect to the opening 28, form an inlet 34 together with the opening 28, wherein the ratio of the total opening area of the inlet 34 to the cross-sectional area of the cavity at the outlet 32 can be 1.5:1 to 2.5:1. Accordingly, advantageous pressure differences and volume flow ratios can be achieved for the respective rows of holes 14, 16, 18 in the distal area of the cannula, such that from a physiological point of view a more advantageous conveying of the body fluid can be provided.

Although in this embodiment the inlet 34 is arranged at the distal end 12 and the outlet 32 at the proximal end 30 (e.g. in the case of blood collection), these can also be functionally reversed in the case of the supply of body fluids, such that the body fluids can be supplied to a patient via the rows of holes 14, 16, 18 and opening 28 when positive pressure is applied.

Where applicable, all the individual features depicted in the exemplary embodiments may be combined and/or exchanged without leaving the scope of the disclosure.

### LIST OF REFERENCE NUMERALS

10 Proximal direction
12 Distal end
14 Row of holes X
16 Proximal row of holes
18 Distal row of holes
20 Proximal row of holes
22 Distal region
24 Hole
26 Cavity
28 Opening
30 Proximal end
32 Outlet
34 Inlet
A-A Cross-section of row of holes X
B-B Cross-section of proximal row of holes

1-46. (canceled)

47. A cannula for conveying body fluids, the cannula comprising:
a tubular body extending in an axial direction from a proximal end to a distal end, wherein the body defines a continuous inner cavity from the proximal end to the distal end and comprises at least two rows of holes in a distal region of the body, wherein the at least two rows of holes are axially spaced from each other and each comprise at least two holes, wherein the holes in each of the at least two rows of holes open from the inner cavity in a radial direction and are spaced apart from one another in a circumferential direction,
wherein a first row of holes of the at least two rows of holes is arranged distally to a second row of holes of the at least two rows of holes, and wherein a ratio of a first total opening area of holes of the first row of holes to a second total opening area of holes of the second row of holes is within a range of 2:1 to 3:1.

48. The cannula according to claim 47, wherein the second row of holes is arranged proximally adjacent to the first row of holes.

49. The cannula according to claim 47, wherein the at least two rows of holes comprises a third row of holes arranged proximally to the first row of holes.

50. The cannula according to claim 47, wherein the at least two rows of holes comprises one or more additional rows of holes arranged distally to the first row of holes.

51. The cannula according to claim 50, wherein the one or more additional rows of holes comprises a single row of holes.

52. The cannula according to claim 50, wherein a ratio of a total opening area of the holes of at least one of the one or more additional rows of holes to the first total opening area of the holes of the first row of holes is 1:1 to 1:1.5.

53. The cannula according to claim 52, wherein the one or more additional rows of holes comprises a single row of holes.

54. The cannula according to claim 50, wherein at least one of the one or more additional rows of holes comprises four holes and/or substantially circularly formed holes.

55. The cannula according to claim 47, wherein the at least two rows of holes comprises two to five rows of holes.

56. The cannula according to claim 47, wherein the distal region of the body extends from the distal end of the body in the proximal direction 30 to 60 cm.

57. The cannula according to claim 47, wherein each of the at least two rows of holes comprises two to six holes.

58. The cannula according to claim 47, wherein a number of holes of each of the at least two rows of holes decreases from the distal end to the proximal end.

59. The cannula according to claim 47, wherein each row of the at least two rows of holes has a number of holes that is equal to a number of holes of a row that is proximally adjacent to each row of the at least two rows of holes.

60. The cannula according to claim 47, wherein each row of the at least two rows of holes has a number of holes that is greater than a number of holes of a row that is proximally to each row of the at least two rows of holes.

61. The cannula according to claim 47, wherein the first row of holes comprises three holes and/or at least one row of one or more additional rows of holes arranged distally to the first row of holes comprises two holes.

62. The cannula according to claim 61, wherein the at least one further row of holes comprises the second row of holes.

63. The cannula according to claim 47, wherein the holes of the at least two rows of holes are substantially equally spaced apart in the axial direction towards the distal end.

64. The cannula according to claim 47, wherein the holes of the at least two rows of holes are substantially circular, ellipsoidal, and/or elongated.

65. The cannula according to claim 47, wherein the holes of the at least two rows of holes are equally shaped.

66. The cannula according to claim 47, wherein the holes of the at least two rows of holes are differently shaped.

67. The cannula according to claim 47, wherein the holes of the first row of holes are formed as oblong holes.

68. The cannula according to claim 47, wherein the at least two rows of holes comprises a third row of holes arranged proximally to the second row of holes, wherein holes of the third row of holes are substantially circular.

69. The cannula according to claim 68, wherein the second row of holes are proximally adjacent to the first row of holes, and wherein the second row of holes comprises circular holes.

70. The cannula according to claim 47, wherein the holes of the at least two rows of holes are rounded towards the outer surface of the body and/or towards an inner wall of the body defining the inner cavity.

71. The cannula according to claim 47, wherein the holes of at least one row of the at least two rows of holes are arranged symmetrically or asymmetrically with respect to the longitudinal axis of the body.

72. The cannula according to claim 47, wherein the holes of at least one row of the at least two rows of holes are uniformly spaced apart from each other in the circumferential direction.

73. The cannula according to claim 47, wherein the body comprises at least one opening at the distal end, wherein the opening opens in the axial direction.

74. The cannula according to claim 73, wherein the at least one opening comprises a single opening which substantially corresponds to a cross-sectional area of the inner cavity.

75. The cannula according to claim 47, wherein the at least two rows of holes comprises at least three rows of holes, and wherein each pair of adjacent rows of holes is differently spaced in the axial direction than any other pair of adjacent rows of holes.

76. The cannula according to claim 47, wherein a first distance between the first row of holes and the second row of holes is about 45 mm to about 60 mm and/or wherein a ratio of the first distance to a second distance between the row of holes and the distal end of the body is 1:1.2 to 1:1.4.

77. The cannula according to claim 47, wherein a first distance between the first row of holes and the distal end of the body is about 60 mm to about 80 mm and/or wherein a ratio of the first distance to a second distance between a most proximally located row of holes of the at least two rows of holes and the distal end of the body is 1:1.65 to 1:1.85.

78. The cannula according to claim 47, wherein the at least two rows of holes comprises one or more additional rows of holes arranged distally to the first row of holes, and wherein a first distance between the first row of holes and a row of holes arranged adjacent to the first row of holes in the distal direction is about 28 mm to about 43 mm and/or wherein a ratio of the first distance to a distance between the first row of holes and the distal end of the body is 1:1.8 to 1:2.2.

79. The cannula according to claim 47, wherein the body comprises an opening at the distal end of the body, wherein the opening opens in the axial direction, and wherein the opening and the holes define an inlet and the proximal end of the body defines an outlet.

80. The cannula according to claim 79, wherein a ratio of a total opening area of the inlet to a cross-sectional area of the inner cavity at the outlet is 1.5:1 to 2.5:1.

81. The cannula according to claim 79, wherein a ratio of a total opening area of the holes of the at least two rows of holes to a cross-sectional area of the inner cavity at the outlet is 1.5:1 to 2:1.

82. The cannula according to claim 47, wherein the first total opening area of the first row of holes is 85% to 95% of a cross-sectional area of the inner cavity at the distal end of the body.

83. The cannula according to claim 47, wherein the second total opening area of the second row of holes of the at least two rows of holes is 30% to 40% of a cross-sectional area of the inner cavity at the distal end of the body.

84. The cannula according to claim 47, wherein the at least two rows of holes comprises one or more additional rows of holes arranged distally to the first row of holes, and wherein a total opening area of the at one or more additional rows of holes is 75% to 85% of a cross-sectional area of the inner cavity at the distal end of the body.

85. The cannula according to claim 47, wherein the proximal end of the body defines an outlet which is fluidically couplable to a circulatory support system, an oxygenator, and/or a pump.

86. The cannula according to claim 85, wherein the cannula is adapted such that, in the inserted state, the cannula lies in an inferior vena cava, wherein the first row of holes is arranged such that the holes of the first row of holes open at a level of the atrium of the right half of the heart.

87. The cannula according to claim 86, wherein, in the inserted state, the second row of holes is arranged such that the holes of the second row of holes open at a level of the inferior vena cava.

88. The cannula according to claim 86, wherein the at least two rows of holes comprises one or more additional rows of holes arranged distally to the first row of holes, wherein, in the inserted state, the one or more additional rows of holes are arranged such that the holes of the one or more additional rows of holes open at a level of a superior vena cava.

89. A circulatory support system comprising:
a cannula comprising:
a tubular body extending in an axial direction from a proximal end to a distal end, wherein the body defines a continuous inner cavity from the proximal end to the distal end and comprises at least two rows of holes in a distal region of the body, wherein the at least two rows of holes are axially spaced from each other and each comprise at least two holes, wherein the holes in each of the at least two rows of holes open from the inner cavity in a radial direction and are spaced apart from each another in the circumferential direction,
wherein a first row of holes of the at least two rows of holes is arranged distally to a second row of holes of the at least two rows of holes, and wherein a ratio of a first total opening area of holes of the first row of holes to a second total opening area of holes of the second row of holes is within a range of 2:1 to 3:1.

90. A method of using a cannula comprising a tubular body extending in an axial direction from a proximal end to a distal end, wherein the body defines a continuous inner cavity from the proximal end to the distal end and comprises at least two rows of holes in a distal region of the body, wherein the at least two rows of holes are axially spaced from each other and each comprise at least two holes, wherein the holes in each of the at least two rows of holes open from the inner cavity in a radial direction and are spaced apart from each another in the circumferential direction, wherein a first row of holes of the at least two rows of holes is arranged distally to a second row of holes of the at least two rows of holes, and wherein a ratio of a first total opening area of holes of the first row of holes to a second total opening area of holes of the second row of holes is within a range of 2:1 to 3:1, the method comprising:
inserting the cannula through an inguinal vein of a patient;
advancing the cannula such that the distal region is advanced into a inferior vena cava of the patient; and
placing the cannula such that the first row of holes is positioned at a level of a right half of a heart of the patient.

91. The method according to claim 90, wherein placing the cannula comprises placing the cannula in such a way that the holes of the first row of holes open at a level of the right atrium.

92. The method according to claim 90, wherein the placing the cannula comprises placing the cannula in such a way that the holes of the second row of holes open at a level of the inferior vena cava.

93. The method according to claim 90, wherein the at least two rows of holes comprises one or more additional rows of holes arranged distally to the first row of holes, and wherein placing the cannula comprises placing the cannula in such a way that the holes of the one or more additional rows of holes open at a level of a superior vena cava.
